(19) United States
(12) Patent Application Publication
(10) Pub. No.: US 2026/0001078 A1
(43) Pub. Date: Jan. 1, 2026

(54) SINGLE CELL PRINTER AND CELL PRINTING METHOD

(71) Applicant: WUHAN OBINL BIOTECH CO.,LTD., Wuhan (CN)

(72) Inventor: XIN YANG, Wuhan (CN)

(21) Appl. No.: 19/210,248

(22) Filed: May 16, 2025

Related U.S. Application Data
(63) Continuation of application No. PCT/CN2024/144272, filed on Dec. 31, 2024.

(30) Foreign Application Priority Data
Jun. 28, 2024 (CN) 2024108619954

(51) Int. Cl. B01L 3/00

(52) U.S. Cl. CPC B01L 3/502761; B01L 3/502715; B01L 2200/028; B01L 2200/0652; B01L 2200/141; B01L 2300/0645; B01L 2400/0487

(57) ABSTRACT

The present disclosure provides a single cell printer and a cell printing method. The single cell printer includes a sampling device, a jetting device, an optical detection device, a sorting device and a control device. A liquid inlet of the jetting device is connected to an outlet of the sampling device, and the cell droplet is jetted through a liquid outlet of the jetting device. The optical detection device obtains droplet image information of the cell droplet in the jetting device. The sorting device is provided at the liquid outlet. The control device controls the jetting device to form the cell droplet and sort it into a preset well plate.

### CROSS-REFERENCE TO RELATED APPLICATIONS

The present disclosure claims priority of the Chinese Patent application No. 2024108619954 entitled “SINGLE CELL PRINTER AND CELL PRINTING METHOD” filed on Jun. 28, 2024, to the China National Intellectual Property Administration, the entire contents of which are incorporated herein by reference.

### FIELD

The present disclosure relates to the technical field of microfluidics, and in particularly to a single cell printer and a cell printing method.

### BACKGROUND

With the continuous development of cell therapy and gene therapy technologies, cell-related research has gradually become a research hotspot in the current medical and the entire life science fields. Cell culture has become a very important technology in scientific research, and cell sorting and purification are the keys to obtaining relatively pure target cells.

Single cell sorting can achieve the capture, separation and analysis of any single type of cells or rare cells (such as circulating tumor cells, endothelial cells, stem cells, and related cells in blood and bone marrow, etc.) in the suspension system. Most of the existing single cell sorting methods adopt the separation and dilution method. By diluting the cell suspension multiple times, single cells are dispersed in the culture medium, and the cell concentration is continuously reduced, ultimately achieving single cell sorting. This method requires a great deal of time, manpower and material resources, has a high cost, a low sorting efficiency, and may result in non-single cell sorting.

In addition, flow cytometer is also a common cell sorting technology. This technology combines the specificity of fluorescently labeled cells to achieve the sorting of a single cell. However, during the sorting process, cross contamination between cells may occur, and strict sample handling and instrument cleaning measures are required. At the same time, during the sorting process of the flow cytometer, cells may be exposed to the damage caused by mechanical stress and photodamage from fluorescent exciters, which may affect the state of the cells. The existing cell sorting methods only screen out droplets containing single cells into the well plate. If the cells themselves do not meet the requirements of the experiment during the subsequent cell culture process, it will interfere with the experimental results and affect the efficiency of the experiment.

### SUMMARY

This application provides a single cell printer and a cell printing method, aiming to solve the problems that may occur during the cell sorting process of existing single cell printers. There may be cross contamination between cells, and the need for strict sample handling and instrument cleaning measures. At the same time, during the sorting process of the flow cytometer, cells may be exposed to the damage caused by mechanical stress and photodamage from fluorescent exciters, which may affect the state of the cells. The existing cell sorting methods only screen out droplets containing single cells into the well plate. If the cells themselves do not meet the requirements of the experiment during the subsequent cell culture process, it will interfere with the experimental results and affect the efficiency of the experiment.

In the first aspect, this application provides a single cell printer, the single cell printer includes:
a sampling device, the sampling device stores cell suspension to be sorted;
a jetting device, a liquid inlet of the jetting device is connected to an outlet of the sampling device, the jetting device is used to control a vibration of the cell suspension to form a cell droplet to be sorted, and make the cell droplet jet through a liquid outlet of the jetting device;
an optical detection device, the optical detection device is provided facing the jetting device and is used to obtain droplet image information of the cell droplet to be sorted in the jetting device;
a sorting device, the sorting device is provided at the liquid outlet;
a control device, the control device is electrically connected to the jetting device, the optical detection device and the sorting device, and is used to control the jetting device to form the cell droplet, obtain the droplet image information detected by the optical detection device, and determine whether the cell droplet is a target droplet according to the droplet image information, so as to sort the target droplet into a preset well plate; wherein the target droplet is the cell droplet containing a preset number of cells that meet a preset cell condition.

In the second aspect, this application provides a cell printing method, the method is applied to a single cell printer provided in any embodiment of this application, this method includes: controlling a jetting device of the single cell printer to form a cell droplet;
obtaining droplet image information of the cell droplet in the jetting device collected by an optical detection device of the single cell printer;
determining whether the cell droplet is a target droplet according to the droplet image information, the target droplet is the cell droplet containing a preset number of cells that meet a preset cell condition; and
if the cell droplet is the target droplet, controlling the sorting device to sort the target droplet into a preset well plate to complete the printing of the target droplet.

This application provides a single cell printer, includes: a sampling device, a jetting device, an optical detection device, a sorting device and a control device. The sampling device stores cell suspension to be sorted. A liquid inlet of the jetting device is connected to an outlet of the sampling device; the jetting device is used to control a vibration of the cell suspension to form a cell droplet to be sorted, and make the cell droplet jet through a liquid outlet of the jetting device. The optical detection device is provided facing the jetting device and is used to obtain droplet image information of the cell droplet to be sorted in the jetting device. The sorting device is provided at the liquid outlet. The control device is electrically connected to the jetting device, the optical detection device and the sorting device, and is used to control the jetting device to form the cell droplet, obtain the droplet image information detected by the optical detection device, and determine whether the cell droplet is a target droplet according to the droplet image information, so as to sort the target droplet into a preset well plate; wherein the target droplet is the cell droplet containing a preset number of cells that meet a preset cell condition.

The provided single cell printer controls the jetting device via the control device, making the cell suspension in the jetting device vibrate to form a cell droplet to be sorted and then jetted, and controls the optical detection device to capture droplet image information of the cell droplet to be sorted. The control device can control the sorting device to sort the target droplet into a preset well plate when it determines that the cell droplet is a target droplet according to the droplet image information. When the provided single cell printer is used in the process of cell sorting, it can reduce the damage to cells and improve the accuracy of the experiment. At the same time, the provided single cell printer can sort out the target droplet of a single cell that meets the experimental requirements without affecting the cell state, improving the sorting accuracy and efficiency of single cell sorting, reducing the equipment cost of the single cell printer, and enabling the single cell printer to be widely applied and popularized in high-throughput sorting applications.

### BRIEF DESCRIPTION OF THE DRAWINGS

In order to clearly illustrate the technical solutions in the embodiments of this application, the following will briefly introduce the drawings used in the description of the embodiments. Apparently, the drawings in the following description are some embodiments of this application. For those of ordinary skill in the art, without making any creative efforts, other drawings can be obtained based on these drawings.

FIG. 1 is a structural schematic diagram of a single cell printer provided in an embodiment of this application.

FIG. 2 is a structural schematic diagram of a sorting device provided in an embodiment of this application.

FIG. 3 is a structural schematic diagram of another sorting device provided in an embodiment of this application.

FIG. 4 is a schematic diagram of the workflow of the cell detection model provided in an embodiment of this application.

FIG. 5 is a structural schematic diagram of a sampling device provided in an embodiment of this application.

FIG. 6 is a structural schematic diagram of a jetting device provided in an embodiment of this application.

FIG. 7 is a schematic flowchart of a cell printing method provided in an embodiment of this application.

FIG. 8 is a structural schematic diagram of a control device provided in an embodiment of this application.

### Explanation of Main Components and Symbols

10, single cell printer; 11, sampling device; 111, sample introduction module; 1111, first pipeline; 1112, second pipeline; 112, mixing module; 1121, second air pump; 1122, second solenoid valve; 12, jetting device; 121, substrate; 1211, cell liquid cavity; 1212, first substrate; 1213, second substrate; 122, first electrode; 123, piezoelectric substrate; 124, second electrode; 13, optical detection device; 14, sorting device; 141, pipetting device; 1411, first air pump; 1412, first solenoid valve; 1413, positive electrode; 142, waste liquid collection area; 143, motion platform; 15, control device; 20, cell droplet; 30, preset well plate.

It should be understood that the above general description and the following detailed description are merely exemplary and explanatory, and shall not limit this application.

### DETAILED DESCRIPTION

The technical solution of embodiments of this application is clearly and completely described in detail in connection with the accompanying drawings. Apparently, described embodiments are some embodiments of this application, not all embodiments. Based on embodiments of this application, all other embodiments obtained by those of ordinary skill in the art without creative efforts are within the scope of this application.

The flowcharts shown in the drawings are merely for illustrative purposes. They do not necessarily include all the content and operations/steps, nor do they have to be executed in the described order. For example, some operations/steps can be further split, combined, or partially merged, so the actual execution order may change according to the actual situation.

It should be understood that the terms used in the specification of this application are merely for the purpose of describing specific embodiments and are not intended to limit this application. As used in the specification of this application and the appended claims, unless the context clearly dictates otherwise, the singular forms “a”, “an”, and “the” are intended to include the plural forms.

It should be understood that, in order to clearly describe the technical solutions of the embodiments of this application, in the embodiments of this application, terms such as “first” and “second” are used to distinguish identical or similar items with basically the same functions and effects. For example, the first data and the second data are only used to distinguish different data, and there is no limitation on their sequence. Those skilled in the art can understand that terms such as “first” and “second” do not limit the quantity or the execution order, and terms such as “first” and “second” do not necessarily mean that they are different.

It should be further understood that the term “and/or” used in the specification and the claims of the present disclosure refers to any combination and all possible combinations of one or more of the items listed in association, and includes these combinations.

The following is a detailed description of some embodiments of this application in connection with the accompanying drawings. In no conflicting situations, the embodiments and features within the embodiments described below can be combined with each other.

With the continuous development of cell therapy and gene therapy technologies, cell-related research has gradually become a research hotspot in the current medical and the entire life science fields. Cell culture has become a very important technology in scientific research, and cell sorting and purification are the keys to obtaining relatively pure target cells.

Single cell sorting can achieve the capture, separation and analysis of any single type of cells or rare cells (such as circulating tumor cells, endothelial cells, stem cells, and related cells in blood, bone marrow, etc.) in the suspension system. Most of the existing single cell sorting methods adopt the separation and dilution method. By diluting the cell suspension multiple times, single cells are dispersed in the culture medium, and the cell concentration is continuously reduced, ultimately achieving single cell sorting. This method requires a great deal of time, manpower and material resources, has a high cost, a low sorting efficiency, and may result in non-single cell sorting.

In addition, flow cytometer is also a common cell sorting technology. This technology combines the specificity of fluorescently labeled cells to achieve the sorting of a single cell. However, during the sorting process, cross contamination between cells may occur, and strict sample handling and instrument cleaning measures are required. At the same time, during the sorting process of the flow cytometer, cells may be exposed to the damage caused by mechanical stress and photodamage from fluorescent exciters, which may affect the state of the cells. The existing cell sorting methods only screen out droplets containing single cells into the well plate. If the cells themselves do not meet the requirements of the experiment during the subsequent cell culture process, it will interfere with the experimental results and affect the efficiency of the experiment.

Single cell printers may have some defects, including but not limited to:
1. Technical limitations: Single cell sorting technology may be subject to technical limitations, such as limitations in sorting accuracy and sorting efficiency. Current technologies may not fully meet the needs of all application scenarios.
2. High cost: Single cell printers usually require expensive equipment and consumables, resulting in a high cost, which limits their popularization and application in some laboratories or institutions.
3. Complex operation: Operating a single cell printer may require professional skills and training, as it involves technologies such as fluid mechanics, optics, and computer control that require certain professional knowledge.
4. Sample damage: During the sorting process, cells may be mechanically or optically damaged, leading to a decrease in sample quality or cell death, affecting the accuracy of experimental results.
5. Long processing time: Sorting a large number of samples may take a long time, which limits the application of single cell sorting systems in high-throughput experiments.
6. Data processing challenges: The amount of data generated by single cell printers may be large, and complex data processing and analysis are required to extract useful information from the massive data, which may be a challenge.
7. Difficulty in handling heterogeneity: The heterogeneity of cell populations may lead to difficulties in the sorting process, as different types of cells may have similar phenotypic characteristics, making it difficult to accurately sort the target cells.
8. Potential cross contamination: During the single cell sorting process, there is a risk of cross contamination, that is, cells sorted from one sample may be mis-placed into another sample, affecting the reliability of experimental results.

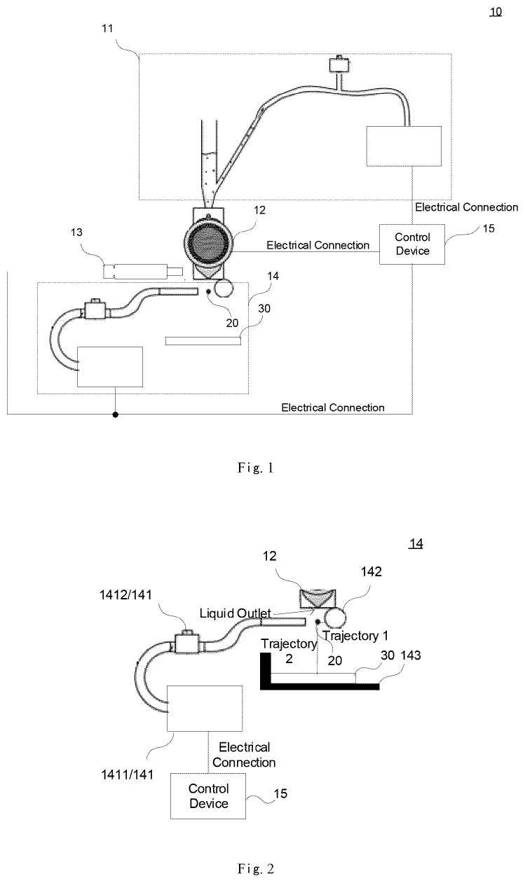

To address the aforementioned problems, please refer to FIG. 1, FIG. 1 is a structural schematic diagram of a single cell printer 10 provided in an embodiment of this application. As shown in FIG. 1, the provided single cell printer 10 includes: a sampling device 11, a jetting device 12, an optical detection device 13, a sorting device 14 and a control device 15. The sampling device 11 stores cell suspension to be sorted. A liquid inlet of the jetting device 12 is connected to an outlet of the sampling device 11; the jetting device 12 is used to control a vibration of the cell suspension to form a cell droplet 20 to be sorted, and make the cell droplet 20 jet through a liquid outlet of the jetting device. The optical detection device 13 is provided facing the jetting device 12 and is used to obtain droplet image information of the cell droplet 20 to be sorted in the jetting device 12. The sorting device 14 is provided at the liquid outlet. The control device 15 is electrically connected to the jetting device 12, the optical detection device 13 and the sorting device 14, and is used to control the jetting device 12 to form the cell droplet, obtain the droplet image information detected by the optical detection device 13, and determine whether the cell droplet 20 is a target droplet according to the droplet image information, so as to sort the target droplet into a preset well plate 30; wherein the target droplet is the cell droplet containing a preset number of cells that meet a preset cell condition.

In an embodiment, the control device 15 controls the jetting device 12, making the cell suspension in the jetting device 12 vibrate to form a cell droplet 20 to be sorted and then jetted, and controls the optical detection device 13 to capture droplet image information of the cell droplet 20 to be sorted. The control device 15 can control the sorting device 14 to sort the target droplet into a preset well plate 30 when it determines that the cell droplet 20 is a target droplet according to the droplet image information. When the provided single cell printer 10 is used in the process of cell sorting, it can reduce the damage to cells and improve the accuracy of the experiment. At the same time, the provided single cell printer 10 can sort out the target droplet containing a preset number of cells that meet the experimental requirements without affecting the cell state, improving the sorting accuracy and efficiency of single cell sorting, reducing the equipment cost of the single cell printer 10, and enabling the single cell printer 10 to be widely applied and popularized in high-throughput sorting applications.

In some embodiments, the preset number can be 1, or any number greater than 1. The provided single cell printer 10 can sort out the target droplet containing a single cell or a plurality of cells according to the experimental requirements, thereby expanding the applicable range of the provided single cell printer 10.

In some embodiments, the preset cell condition includes: the cell circularity of at least one cell in the cell droplet 20 is within the preset circularity range, and the size of the cell in the cell droplet is within the preset size range.

The preset circularity range of the cell circularity is generally from 0.00 to 1.00. The larger the value of the cell circularity, the rounder the cell within the cell droplet 20. When the cell circularity is 1.00, it is a standard circle. In this application, by selecting the cell circularity and size of the cell droplet 20, for example, the preset circularity is 0.8, and the preset size range can be a diameter of 10-30 μm, the target droplet of a single cell that meet the experimental requirements can be sorted out. At the same time, the preset circularity and preset size can be adjusted according to the experimental requirements, and the values of the preset circularity range and the preset size range are not limited in the embodiments of this application.

It should be noted that, in some embodiments, whether a droplet is a target droplet can also be determined by setting a range based on parameters such as the success of cell printing (whether the target droplet is jetted or not), cell area, cell perimeter, aspect ratio, circularity, etc. The specific types of parameters selected can be set according to actual needs, and the embodiments of this application do not impose any limitations on this.

In some embodiments, please refer to FIG. 2. FIG. 2 is a structural schematic diagram of a sorting device 14 provided in an embodiment of this application. As shown in FIG. 2, the sorting device 14 includes a pipetting device 141 and a waste liquid collection area 142. The pipetting device 141 is provided at the liquid outlet, and the waste liquid collection area 142 is also provided at the liquid outlet. Wherein the control device 15 is electrically connected to the pipetting device 141. The control device 15 is used to obtain the number of cells and cell parameter information according to the droplet image information, so as to control the pipetting device 141 to move the cell droplet 20 whose number of cells is not 1 and whose cell parameter information does not meet the preset cell condition to the waste liquid collection area 142.

During the cell sorting process of the single cell printer 10, when the control device 15 determines, based on the droplet image information, that the cell droplet 20 jetted from the liquid outlet of the jetting device 12 does not meet the preset cell condition, the control device 15 can control the pipetting device 141 to promptly move the cell droplet 20 to the waste liquid collection area 142. At the same time, it directly sorts the cell droplet 20 that meets the preset cell condition into the preset well plate 30. Thus, the provided single cell printer 10 can quickly sort the cell droplet 20 containing a single cell.

In an embodiment, the waste liquid collection area 142 can be a cleaning cotton or a waste liquid groove. The embodiments of this application do not impose any limitations on the type of the waste liquid collection area 142.

In an embodiment, as shown in FIG. 2, the pipetting device 141 includes a first air pump 1411. The first air pump 1411 is provided at the liquid outlet opposite to the waste liquid collection area 142. Wherein the control device 15 is electrically connected to the first air pump 1411. The control device 15 is used to control the first air pump 1411 to turn on, making the first air pump 1411 blow gas towards the cell droplet 20 jetted by the jetting device 12, so that the cell droplet 20 moves to the waste liquid collection area 142. When the first air pump 1411 is turned off, the cell droplet 20 is jetted into the preset well plate 30.

When the control device 15 controls the first air pump 1411 to turn on, air will be blown towards the waste liquid collection area 142 at the jet port. As a result, the cell droplet 20 jetted through the jet port will be blown into the waste liquid collection area.

It should be noted that, in some embodiments, the pipetting device 141 further includes a first solenoid valve 1412. The first air pump 1411 is connected to the first interface of the first solenoid valve 1412 via a first pipeline. The second interface of the first solenoid valve 1412, which is provided opposite to the first interface, is connected to a second pipeline. The first solenoid valve 1412 is electrically connected to the control device 15. When the control device 15 controls the first interface and the second interface of the first solenoid valve to be connected, the first air pump 1411 can blow gas through the first solenoid valve 1412 towards the cell droplet 20 jetted by the jetting device 12.

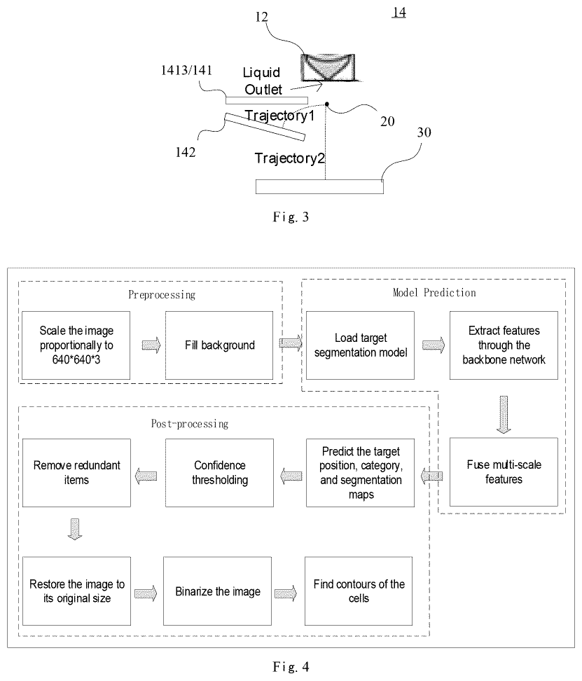

In an embodiment, as shown in FIG. 3, FIG. 3 is a structural schematic diagram of another sorting device 14 provided in an embodiment of this application. The polarity of the cell suspension is negative; the pipetting device 141 includes a positive electrode 1413. The positive electrode 1413 is provided at the liquid outlet and is located above the waste liquid collection area 142. The positive electrode 1413 is electrically connected to the control device 15. When the control device 15 controls the positive electrode 1413 to be turned on, the cell droplet 20 jetted by the jetting device 12 will move towards the direction of the positive electrode 1413 and be jetted into the waste liquid collection area 142 (as shown by trajectory 1 in FIG. 3); when the positive electrode is turned off, the cell droplet is jetted into the preset well plate (as shown by trajectory 2 in FIG. 3).

In some embodiments, the sorting device 14 includes a liquid suction device. The liquid suction device is provided at the liquid outlet and is electrically connected to the control device 15. When the control device 15 controls the liquid suction device to turn on, the liquid suction device will suck away the cell droplet 20 jetted by the jetting device 12. When the control device 15 controls the liquid suction device to turn off, the cell droplet 20 is jetted into the preset well plate.

The liquid suction device can be the first air pump 1411 as shown in FIG. 2. In this case, when the first air pump 1411 is turned on, it sucks air towards the liquid outlet of the jetting device 12, thereby sucking away the cell droplet 20 that does not meet the preset cell condition. In some embodiments, as shown in FIG. 2, the sorting device 14 further includes a motion platform 143. The preset well plate 30 is placed on the motion platform 143, and the motion platform 143 is electrically connected to the control device 15. When the cell parameter information meets the preset cell condition, the control device 15 determines a target groove in the multiple grooves of the preset well plate 30 for the target droplet to be jetted into. Then, according to the coordinate information of the target groove, the control device 15 controls the motion platform 143 to move the preset well plate 30 so that the liquid outlet of the jetting device 12 is aligned with the target groove. With the arrangement of the motion platform 143, the corresponding target groove can be quickly aligned each time a cell droplet 20 is jetted, which improves the efficiency of cell sorting.

In an embodiment, the motion platform 143 can be an X/Y dual-axis motion platform or a single-axis motion platform. The embodiments of this application do not impose any limitations on the type of the motion platform 143.

In an embodiment, the liquid suction device includes a preset liquid suction area. The preset liquid suction area is provided on the motion platform 143. When the cell droplet 20 is not the target droplet, the control device 15 controls the motion platform 143 to move the preset liquid suction area so that the liquid outlet of the jetting device 12 is aligned with the preset liquid suction area. The preset liquid suction area can be a cleaning cotton or another well plate. The embodiments of this application do not impose any limitations on the type of the preset liquid suction area.

In some embodiments, the motion platform 143 is connected to the jetting device 12 via fasteners such as clamps, so that the liquid outlet of the jetting device 12 can be controlled to align with the waste liquid collection area 142 or the preset well plate 30.

In some embodiments, the control device includes a Microcontroller Unit (MCU), or a terminal device equipped with an MCU.

In some embodiments, as shown in FIG. 4, FIG. 4 is a schematic diagram of the workflow of the cell detection model provided in an embodiment of this application. The control device 15 stores a preset cell detection model, and the control device 15 is used to input the droplet image information into the cell detection model so as to obtain the cell number information and the cell parameter information corresponding to the droplet image information. Wherein the cell number information is used to determine the number of cells in the cell droplet.

The provided cell detection model mainly adopts the YOLOv8 segmentation model based on visual imaging. As shown in FIG. 4, this model has a strong feature extraction ability and can better capture the detailed features of the target. Meanwhile, the YOLOv8 algorithm uses a one-stage method for object detection, which has a relatively fast detection speed. Therefore, this algorithm can meet the requirements of both high recognition rate and fast printing for the single cell printer. Before the droplet image information is input into the model, it needs to be scaled proportionally and the background needs to be filled to adapt to the input size of the model. The internal calculations of the model mainly include feature extraction and feature fusion. Finally, the position, category, and segmentation map of the target are output. Targets with low probabilities are removed through confidence thresholding, and redundant items are removed through maximum suppression. Then, the output segmentation map is restored to its original size. By binarizing the image using a threshold, the contours of the cells can be found.

It should be noted that, in some embodiments, the currently trained latest model has accumulated 3,000 pieces of labeled droplet image information. The training results show that the recognition rate of cells can reach 97%.

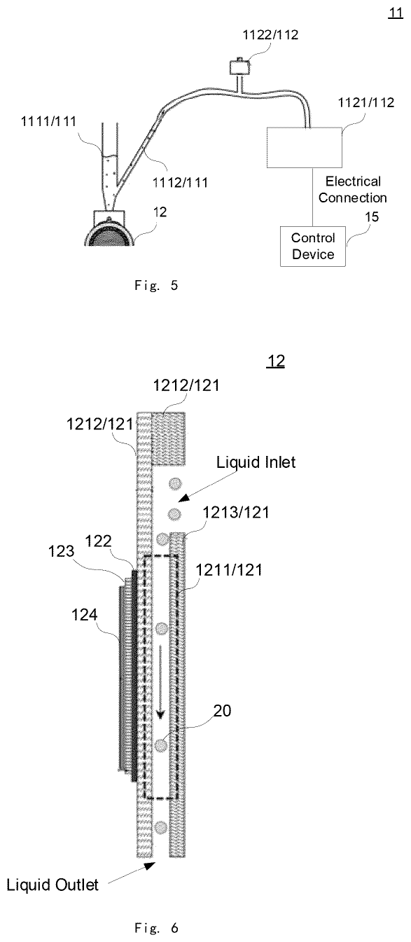

In some embodiments, please refer to FIG. 5. FIG. 5 is a structural schematic diagram of a sampling device 11 provided in an embodiment of this application. The sampling device 11 includes a sample introduction module 111 and a mixing module 112. The sample introduction module 111 includes a first pipeline 1111 and a second pipeline 1112. The inlet of the first pipeline 1111 is used to input the cell suspension, and the outlet of the first pipeline 1111 is connected to the liquid inlet of the jetting device 12. One side of the first pipeline 1111 is connected to the outlet of the second pipeline 1112. The mixing module 112 is connected to the inlet of the second pipeline 1112 and is also electrically connected to the control device 15. The control device 15 is used to determine the number of cells in the cell suspension in the jetting device 12 according to the droplet image information, and control the mixing module 112 to perform reciprocating gas suction at the outlet of the second pipeline according to the number of cells, so as to ensure that the cell suspension flowing into the jetting device 12 is in a well-mixed state. Therefore, the single cell printer 10 provided by this application can avoid the problems that since the cells in the cell suspension are very prone to sedimentation, the cell sedimentation may cause a blockage to form in the jetting device 12, thus affecting the normal operation of the cell sorting process, and the efficiency and accuracy of the final optical detection results of the sorted cell droplet.

In an embodiment, as shown in FIG. 5, the mixing module 112 includes a second air pump 1121. The second air pump 1121 is connected to the inlet of the second pipeline 1112, and the control device 15 is electrically connected to the second air pump 1121. The control device 15 is configured to determine the control parameter for the second air pump 1121 based on the number of cells in the cell suspension within the jetting device 12. Then the control device 15, according to the control parameter, controls the second air pump 1121 to perform reciprocating gas suction at the outlet of the second pipeline 1112, ensuring that the cell suspension flowing into the jetting device 12 remains in a well-mixed state.

In an embodiment, the control parameter includes the suction power of the second air pump. When the number of cells is less than the preset number, the control device adjusts the control parameter to reduce the suction power of the second air pump so as to increase the number of cells. When the number of cells is greater than the preset number, the control device adjusts the control parameter to increase the suction power of the second air pump so as to decrease the number of cells. When the number of cells is equal to the preset number, the control device does not adjust the control parameter.

In some embodiments, please refer to FIG. 6, FIG. 6 is a structural schematic diagram of a jetting device 12 provided in an embodiment of this application. The jetting device 12 includes a substrate 121, a first electrode 122, a piezoelectric substrate 123, and a second electrode 124. The substrate 121 includes a cell liquid cavity 1211, which is used to hold the cell suspension to be sorted. The cell liquid cavity 1211 has a liquid inlet and a liquid outlet, and the liquid inlet is connected to the sampling device 11. The optical detection device 13 faces the substrate 121 and is opposite to the cell liquid cavity 1211. The first electrode 122 is provided on the substrate 121 and located outside the cell liquid cavity 1211. The piezoelectric substrate 123 is made of piezoelectric material and is provided on the first electrode 122. The second electrode 124 is provided on the piezoelectric substrate 123, and the polarities of the second electrode 124 and the first electrode 122 are opposite. The second electrode 124 and the first electrode 122 are electrically connected to the control device 15. The control device 15 is used to control the first electrode 122 and the second electrode 124 to drive the piezoelectric substrate 123 to vibrate, so as to transmit acoustic pulse waves in the cell suspension in the cell liquid cavity 1211. This drives the cell suspension to vibrate to form the cell droplet 20 and then make the cell droplet 20 jet through the liquid outlet. Therefore, the provided single cell printer 10 can reduce the damage to cells during the cell sorting process and improve the accuracy of the experiment.

In an embodiment, as shown in FIG. 6, the substrate 121 includes a first substrate 1212 and a second substrate 1213. The first electrode 122 is disposed on the first side surface of the first substrate 1212. The second side surface of the first substrate 1212 is connected to the first side surface of the second substrate 1213 to enclose the cell liquid cavity 1211. The transmittance of the first substrate 1212 is greater than the preset transmittance. The optical detection device 13 faces the first substrate 1212 and is opposite to the cell liquid cavity 1211.

It should be noted that, in some embodiments, the first substrate 1212 can be made of a light-transmitting material such as glass, and the second substrate can be made of silicon. At this time, the light of the optical detection device 13 will enter the cell liquid cavity 1211 through the first substrate 1212 and then be reflected back by the second substrate 1213. If the second substrate 1213 is also made of the light-transmitting material, a receiving device needs to be provided opposite to the second substrate 1213 for the optical detection device.

It should be noted that, in some embodiments, a solution groove is provided on the first side surface of the second substrate 1213. When the second side surface of the first substrate 1212 is connected to the first side surface of the second substrate 1213, the solution groove and the second side surface of the first substrate 1212 form the cell liquid cavity 1211.

It should be noted that, in some embodiments, the liquid inlet is provided on the second side surface of the second substrate 1213; the solution groove includes a liquid outlet groove, and the liquid outlet groove and the second side surface of the first substrate 1212 form the liquid outlet.

It should be noted that, in some embodiments, the length of the first electrode 122 in the flowing direction of the cell suspension is a first preset length, and the length of the piezoelectric substrate 123 in the flowing direction of the cell suspension is a second preset length.

In the solution provided by this application, in the flowing direction of the cell suspension, the first electrode 122 must cover the piezoelectric substrate 123 to ensure that the piezoelectric substrate 123 can vibrate fully. If the first preset length is equal to or slightly smaller than the second preset length, although the solution provided in the embodiments of this application can still be realized, the vibration effect of the piezoelectric substrate 123 on the cell liquid cavity 1211 will be affected.

In an embodiment, the length of the second electrode 124 in the flowing direction of the cell suspension can be less than the second preset length, or can be greater than or equal to the second preset length. The embodiments of this application do not impose any limitations on the length of the second electrode 124 in the flowing direction of the cell suspension.

It should be noted that, in some embodiments, the second preset length is greater than the length of the cell liquid cavity 1211 in the flowing direction of the cell suspension; or, the second preset length is equal to the length of the cell liquid cavity 1211 in the flowing direction of the cell suspension.

It should be noted that, in some embodiments, the cross-sectional shapes of the first electrode 122, the second electrode 124, and the piezoelectric substrate 123 in the flowing direction of the cell suspension are the same as the cross-sectional shape of the cell liquid cavity 1211 in the flowing direction of the cell suspension.

The single cell printer provided in some embodiments of this application controls the jetting device via the control device, making the cell suspension in the jetting device vibrate to form a cell droplet to be sorted and then jetted, and controls the optical detection device to capture droplet image information of the cell droplet to be sorted. The control device can control the sorting device to sort the target droplet into a preset well plate when it determines that the cell droplet is a target droplet according to the droplet image information. When the provided single cell printer is used in the process of cell sorting, there is no external force acting on the cells, reducing the damage to cells and improving the accuracy of the experiment. At the same time, the provided single cell printer can sort out the target droplet with a single cell that meets the experimental requirements without affecting the cell state, improving the sorting accuracy and efficiency of single cell sorting, reducing the equipment cost of the single cell printer, and enabling the single cell printer to be widely applied and popularized in high-throughput sorting applications.

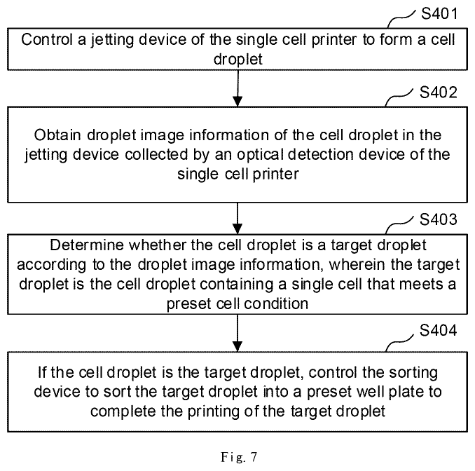

Please refer to FIG. 7, FIG. 7 is a schematic flowchart of a cell printing method provided in an embodiment of this application. The method is applied to the single cell printer provided in any of the embodiments corresponding to FIGS. 1 to 6.

As shown in FIG. 7, the cell printing method includes the following steps.

In step 401, control a jetting device of the single cell printer to form a cell droplet.

In an embodiment, the cell droplet is formed by controlling the jetting device of the single cell printer. For example, by transmitting acoustic pulse waves to the cell liquid cavity, the cell droplet to be sorted can be formed.

In step 402, obtain droplet image information of the cell droplet in the jetting device collected by an optical detection device of the single cell printer.

In an embodiment, by obtaining the droplet image information collected by the optical detection device when the cell droplet is jetted from the jetting device, the state of the cell droplet can be determined.

In step 403, determine whether the cell droplet is a target droplet according to the droplet image information, the target droplet is the cell droplet containing a single cell that meets a preset cell condition.

In an embodiment, when determining that, according to the droplet image information, the cell droplet is a target droplet containing a single cell that meets a preset cell condition, for example, the cell circularity of the cell droplet is greater than the preset circularity and the size of the cell of the cell droplet is greater than the preset size, it can quickly complete the sorting of cell droplets.

In step 404, if the cell droplet is the target droplet, control the sorting device to sort the target droplet into a preset well plate to complete the printing of the target droplet.

In an embodiment, after determining that the cell droplet is a target droplet, the sorting device is used to quickly sort the cell droplet into the target groove corresponding to the preset well plate. This can reduce the damage to the cells during the cell sorting process and improve the accuracy of the experiment. At the same time, the provided single cell printer can sort out the target droplet containing a single cell that meets the experimental requirements without affecting the state of the cells, improving the sorting accuracy and efficiency of single cell sorting, reducing the equipment cost of the single cell printer, and enabling the single cell printer to be widely applied and popularized in high-throughput sorting applications.

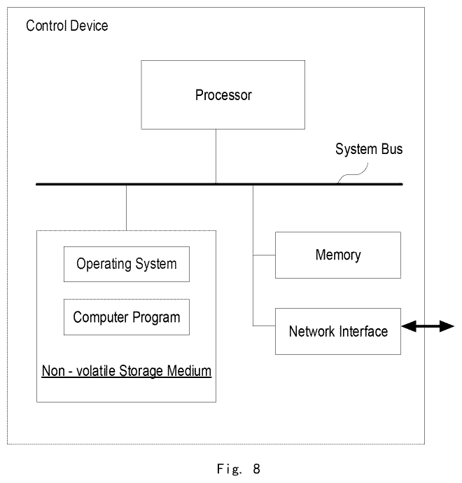

Please refer to FIG. 8, FIG. 8 is a structural schematic diagram of a control device provided in an embodiment of this application.

Please refer to FIG. 8, the control device includes a processor, a memory, and a network interface connected via a system bus. Wherein the memory may include a non-volatile storage medium and an internal memory.

The non-volatile storage medium can store the operating system and a computer program. The computer program includes program instructions. When these program instructions are executed, the processor can be used to execute the cell printing method provided in FIG. 7 and any of its corresponding embodiments.

The processor is used to provide computing and control capabilities, supporting the operation of the entire computer device.

The internal memory provides the environment for the operation of the computer program in the non-volatile storage medium. When the computer program is executed by the processor, it enables the processor to execute the cell printing method provided in FIG. 7 and any of its corresponding embodiments.

The network interface is used for network communication, such as sending out assigned tasks and so on. Those skilled in the art can understand that the structure shown in FIG. 8 is merely a block diagram of some of the structures related to the solution of this application, and does not constitute a limitation on the control device to which the solution of this application is applied. The specific control device may include more or fewer components than those shown in the figure, or combine certain components, or have different component arrangements.

It should be understood that the processor may be a Central Processing Unit (CPU). The processor may also be other general-purpose processor, a Digital Signal Processor (DSP), an Application Specific Integrated Circuit (ASIC), a Field-Programmable Gate Array (FPGA), or other programmable logic device, a discrete gate or a transistor logic device, a discrete hardware component, etc. Wherein the general-purpose processor may be a microprocessor, or the processor may also be any conventional processor, etc.

It should be noted that those skilled in the art can clearly understand that, for the sake of convenience and conciseness of description, the specific working process of the control device described above can refer to the corresponding process in the cell printing method provided in FIG. 7 and any of its corresponding embodiments, and will not be repeated here.

In an embodiment of this application, a computer-readable storage medium is also provided. The computer-readable storage medium stores a computer program, and the computer program includes program instructions. When the processor executes the program instructions, the steps of the cell printing method provided in the above embodiments are implemented. For example, when the computer program is loaded by the processor, the following steps can be executed:
controlling a jetting device of the single cell printer to form a cell droplet;
obtaining droplet image information of the cell droplet in the jetting device collected by an optical detection device of the single cell printer;
determining whether the cell droplet is a target droplet according to the droplet image information, the target droplet is the cell droplet containing a preset number of cells that meet a preset cell condition; and
if the cell droplet is the target droplet, controlling the sorting device to sort the target droplet into a preset well plate to complete the printing of the target droplet.

The specific implementation of each of the above operations can be referred to in the previous embodiments, and will not be repeated here.

The computer-readable storage medium can be an internal storage unit of the aforementioned computer device, such as the hard disk drive or memory of the computer device. The computer-readable storage medium can also be an external storage device of the computer device, such as a pluggable hard disk drive, a smart media card (SMC), a secure digital (SD) card, a flash card equipped on the computer device.

Since the computer program stored in the computer-readable storage medium can execute any of the cell printing methods provided in the embodiments of this application, it can achieve the beneficial effects that can be achieved by any of the cell printing methods provided in the embodiments of this application. For details, refer to the previous embodiments, and they will not be repeated here.

The aforementioned descriptions are merely specific embodiments of this application, but are not intended to limit the protection scope of this application. Any variation or replacement readily figured out by a person skilled in the art within the technical scope disclosed in this application shall fall within the protection scope of this application. Therefore, the protection scope of this application shall be subject to the protection scope of the claims.

What is claimed is:

1. A single cell printer, comprising:
a sampling device, the sampling device stores cell suspension to be sorted;
a jetting device, a liquid inlet of the jetting device is connected to an outlet of the sampling device, the jetting device is used to control a vibration of the cell suspension to form a cell droplet to be sorted, and make the cell droplet jet through a liquid outlet of the jetting device;
an optical detection device, the optical detection device is provided facing the jetting device and is used to obtain droplet image information of the cell droplet to be sorted in the jetting device;
a sorting device, the sorting device is provided at the liquid outlet; and
a control device, the control device is electrically connected to the jetting device, the optical detection device and the sorting device, and the control device is used to control the jetting device to form the cell droplet, obtain the droplet image information detected by the optical detection device, and determine whether the cell droplet is a target droplet according to the droplet image information, so as to sort the target droplet into a preset well plate, wherein the target droplet is the cell droplet containing a preset number of cells that meet a preset cell condition.

2. The single cell printer according to claim 1, wherein the preset cell condition comprises: a cell circularity of at least one cell in the cell droplet is within a preset circularity range, and a size of a cell in the cell droplet is within a preset size range.

3. The single cell printer according to claim 1, wherein the sorting device comprises:
a pipetting device, the pipetting device is provided at the liquid outlet;
a waste liquid collection area, the waste liquid collection area is provided at the liquid outlet;
wherein the control device is electrically connected to the pipetting device; the control device is used to obtain a number of cells and cell parameter information according to the droplet image information, so as to control the pipetting device to move the cell droplet whose number of cells is not the preset number and whose cell parameter information does not meet the preset cell condition to the waste liquid collection area.

4. The single cell printer according to claim 3, wherein the pipetting device comprises a first air pump, the first air pump is provided at the liquid outlet opposite to the waste liquid collection area;
wherein the control device is electrically connected to the first air pump, and is used to control the first air pump to turn on, making the first air pump blow gas towards the cell droplet jetted by the jetting device, so that the cell droplet moves to the waste liquid collection area; when the first air pump is turned off, the cell droplet is jetted into the preset well plate.

5. The single cell printer according to claim 1, wherein the sorting device further comprises:
a motion platform, the preset well plate is provided on the motion platform, and the motion platform is electrically connected to the control device; when the cell parameter information meets the preset cell condition, the control device determines a target groove in multiple grooves of the preset well plate for the target droplet to be jetted into, and then controls the motion platform to move the preset well plate based on coordinate information of the target groove, so that the liquid outlet of the jetting device is aligned with the target groove.

6. The single cell printer according to claim 1, wherein the sampling device comprises:
a sample introduction module, the sample introduction module comprises a first pipeline and a second pipeline; an inlet of the first pipeline is used to input the cell suspension, an outlet of the first pipeline is connected to the liquid inlet of the jetting device, and one side of the first pipeline is connected to an outlet of the second pipeline;
a mixing module, the mixing module is connected to an inlet of the second pipeline and is also electrically connected to the control device;
the control device is used to determine a number of cells in the cell suspension in the jetting device according to the droplet image information, and control the mixing module to perform reciprocating gas suction at the outlet of the second pipeline according to the number of cells, so as to ensure that the cell suspension flowing into the jetting device is in a well-mixed state.

7. The single cell printer according to claim 6, wherein the mixing module comprises:
a second air pump, the second air pump is connected to the inlet of the second pipeline, and the control device is electrically connected to the second air pump;
wherein the control device is configured to determine a control parameter for the second air pump based on the number of cells in the cell suspension within the jetting device, and then control the second air pump to perform reciprocating gas suction at the outlet of the second pipeline according to the control parameter, ensuring that the cell suspension flowing into the jetting device is in the well-mixed state.

8. The single cell printer according to claim 1, wherein the jetting device comprises:
a substrate, the substrate comprises a cell liquid cavity, the cell liquid cavity is used to hold the cell suspension to be sorted, the cell liquid cavity comprises a liquid inlet and a liquid outlet, and the liquid inlet is connected to the sampling device; the optical detection device faces the substrate and is opposite to the cell liquid cavity;
a first electrode, the first electrode is provided on the substrate and located outside the cell liquid cavity;
a piezoelectric substrate, the piezoelectric substrate is made of piezoelectric material and is provided on the first electrode;
a second electrode, the second electrode is provided on the piezoelectric substrate, and polarities of the second electrode and the first electrode are opposite;
wherein the second electrode and the first electrode are electrically connected to the control device; the control device is used to control the first electrode and the second electrode to drive the piezoelectric substrate to vibrate, so as to transmit acoustic pulse waves in the cell suspension in the cell liquid cavity, to drive the cell suspension to vibrate to form the cell droplet and then make the cell droplet jet through the liquid outlet.

9. The single cell printer according to claim 8, wherein a length of the first electrode in a flowing direction of the cell suspension is a first preset length, and a length of the piezoelectric substrate in the flowing direction of the cell suspension is a second preset length.

10. The single cell printer according to claim 9, wherein the first preset length is greater than the second preset length.

11. The single cell printer according to claim 8, wherein cross-sectional shapes of the first electrode, the second electrode, and the piezoelectric substrate in a flowing direction of the cell suspension are the same as a cross-sectional shape of the cell liquid cavity in the flowing direction of the cell suspension.

12. The single cell printer according to claim 10, wherein the second preset length is greater than a length of the cell liquid cavity in the flowing direction of the cell suspension; or, the second preset length is equal to the length of the cell liquid cavity in the flowing direction of the cell suspension.

13. The single cell printer according to claim 8, wherein the substrate comprises:
a first substrate, the first electrode is provided on a first side surface of the first substrate; and
a second substrate, a second side surface of the first substrate is connected to a first side surface of the second substrate to enclose the cell liquid cavity.

14. The single cell printer according to claim 13, wherein a transmittance of the first substrate is greater than a preset transmittance, the optical detection device faces the first substrate and is opposite to the cell liquid cavity.

15. The single cell printer according to claim 13, wherein a solution groove is provided on the first side surface of the second substrate, when the second side surface of the first substrate is connected to the first side surface of the second substrate, the solution groove and the second side surface of the first substrate form the cell liquid cavity.

16. The single cell printer according to claim 15, wherein the liquid inlet is provided on a second side surface of the second substrate; the solution groove comprises a liquid outlet groove, and the liquid outlet groove and the second side surface of the first substrate form the liquid outlet.

17. The single cell printer according to claim 13, wherein the first substrate is made of a light-transmitting material, and the second substrate is made of silicon; the light of the optical detection device enters the cell liquid cavity through the first substrate and then is reflected back by the second substrate.

18. The single cell printer according to claim 13, wherein the first substrate and the second substrate are both made of a light-transmitting material, and a receiving device needs to be provided opposite to the second substrate for the optical detection device.

19. A cell printing method, applied to a single cell printer according to claim 1; the method comprises:
controlling a jetting device of the single cell printer to form a cell droplet;
obtaining droplet image information of the cell droplet in the jetting device collected by an optical detection device of the single cell printer;
determining whether the cell droplet is a target droplet according to the droplet image information, wherein the target droplet is the cell droplet containing a preset number of cells that meet a preset cell condition; and
if the cell droplet is the target droplet, controlling a sorting device to sort the target droplet into a preset well plate to complete a printing of the target droplet.

20. A computer-readable storage medium, wherein the computer-readable storage medium stores a computer program, the computer program comprises program instructions, when the program instructions are executed by a processor, the processor is configured to:
control a jetting device of a single cell printer to form a cell droplet;
obtain droplet image information of the cell droplet in the jetting device collected by an optical detection device of the single cell printer;
determine whether the cell droplet is a target droplet according to the droplet image information, wherein the target droplet is the cell droplet containing a preset number of cells that meet a preset cell condition; and
if the cell droplet is the target droplet, control a sorting device to sort the target droplet into a preset well plate to complete a printing of the target droplet.
